(19) United States
(12) Patent Application Publication
(10) Pub. No.: US 2020/0376037 A1
(43) Pub. Date: Dec. 3, 2020

(54) STEM CELL PREPARATION RESISTING HYPOXIA INJURY, PREPARATION METHOD THEREFOR, AND USE THEREOF IN PREPARATION OF MEDICAMENT FOR TREATING ACUTE MYOCARDIAL INFARCTION

(71) Applicant: SOOCHOW UNIVERSITY, Suchou (CN)

(72) Inventors: Han SHEN, Suzhou (CN); Weiqian CHEN, Suzhou (CN); Zhenya SHEN, Suzhou (CN); Guanghao CUI, Suzhou (CN)

(21) Appl. No.: 16/991,902

(22) Filed: Aug. 12, 2020

Related U.S. Application Data
(63) Continuation of application No. PCT/CN2018/076578, filed on Feb. 12, 2018.

(51) Int. Cl. A61K 35/28; A61P 9/10; C12N 5/0775; C12N 5/00

(52) U.S. Cl. CPC A61K 35/28; C12N 5/0037; C12N 5/0663; A61P 9/10

(57) ABSTRACT

A stem cell agent for anti-hypoxia injury and its preparation method, and its application in the drugs for the therapy of acute myocardial infarction. The preparation method for the stem cell agent for anti-hypoxia injury comprises mixed stem cells and a liquid of lentivirus with Fstl1 over-expression in a medium to obtain the stem cell agent for anti-hypoxia injury. The Fstl1 modified MSCs provided by the present invention have anti-hypoxia injury capability, can improve transplant survival rate, and can improve the cardiac function after infarction by means of direct injection into the cardiac muscle.

This application is a Continuation Application of PCT/CN2018/076578, filed on Feb. 12, 2018, which is incorporated by reference for all purposes as if fully set forth herein.

### TECHNICAL FIELD

The invention belongs to technology for cell drug, more specifically, relates to a stem cell agent for anti-hypoxia injury and its preparation method, and its application in the drugs for the therapy of acute myocardial infarction.

### BACKGROUND TECHNIQUE

Myocardial infarction is a serious cardiovascular disease that endangers human health. With the continuous improvement of people's living standard, the incidence rate of ischemic myocardial infarction is also increasing. Ischemic myocardial infarction can lead to myocardial cell necrosis and scar formation, and then affect cardiac function. At present, drug or device therapy can only relieve symptoms, but can not reverse heart tissue damage. Although heart transplantation can completely improve the heart state, it is difficult to be widely used in clinic due to the shortage of donor sources, immune rejection and expensive therapy costs.

Stem cell transplantation is expected to become an important therapy for myocardial infarction due to its advantages in various aspects. Mesenchymal stem cells (MSCs) are primary adult stem cells with abundant sources and easy to obtain. They mainly exist in bone marrow, fat, umbilical cord, placenta and amniotic fluid. In recent years, MSCs have been widely used in regenerative medicine as seed cells for cell transplantation because of their excellent tissue repair ability. Many studies have confirmed that MSCs mainly rely on paracrine. At present, the bottleneck problem of stem cell transplantation is the low survival rate of stem cell transplantation. Therefore, how to maximize the survival rate of stem cells is a research hotspot in this field. The poor hypoxia microenvironment in myocardial infarction is the main reason for the low survival rate of transplanted stem cells. Therefore, improving the anti hypoxia damage ability of stem cells is the future direction.

Follistatin like 1 (Fsl1) was originally cloned from mice osteoblast MC3T3-E1. It is a secretory extracellular glycoprotein, which does not participate in extracellular matrix structure, but regulates cell physiological activity by changing cell microenvironment at the extracellular level. FSTL1 is an important endogenous active factor that maintains cardiac homeostasis and pathological remodeling, and is widely expressed in the heart. As a marker of left ventricular remodeling in chronic systolic heart failure, FSTL1 can reduce the pathological cardiac hypertrophy caused by pressure overload; however, it is not clear whether Fsl1 can improve the ability of stem cells to resist hypoxia injury and then improve the survival of transplanted cells.

### Technical Problem

The invention discloses a stem cell agent for anti-hypoxia injury and its preparation method, and its application in the drugs for the therapy of acute myocardial infarction.

### Technical Solution

The invention adopted the technical solutions as follows:

A preparation method of stem cell agent for anti-hypoxia injury included the following steps: The stem cells were put into Fsl1 over-expression lentivirus with the culture medium have changed after 10-15 hours, obtained the stem cell agent for anti-hypoxia injury.

The invention also discloses a preparation method of the drug for preventing and therapy of acute myocardial infarction, which comprised the following steps: The stem cells were put into Fsl1 over-expression lentivirus with the culture medium have changed after 10-15 hours, obtained the stem cell agent for anti-hypoxia injury, mixed the stem cell agent for anti-hypoxia injury with the dispersion medium, such as, buffer and normal saline, obtained the drug for preventing or therapy of acute myocardial infarction. The concrete mode of administration can take intramyocardial injection or various ways of blood injection.

Preferably, in the presence of polybrene, the stem cells were put into Fsl1 over-expression lentivirus with the culture medium, to increase the efficiency of virus infection; the final concentration of polybrene was 8 μg/ml.

In the technical solution above, the stem cells were mice bone marrow derived mesenchymal stem cells (MSC). Accorded to the multiplicity of infection, MOI=10 calculate the amount of Fsl1 over-expression lentivirus. In the Fsl1 over-expression lentivirus, the titer of Fsl1 over-expression lentivirus was 10⁷-10⁸ TU/ml; The culture medium was DMEM/F12.

Preferably, selected the male mice C57BL/6J at 2-3 weeks old, which killed by cervical dislocation. Then mice were immersed in 75% alcohol for 10 minutes. Under sterile conditions, removed the tow lower limbs, which were immersed in serum free basic medium DMEM/F12. Remove the surface muscles of femur and tibia; Imbibed with 1 ml injector the serum free basic medium DMEM/F12 which were added penicillin and streptomycin, and then wash the marrow cavity; After wash the marrow all out, centrifugation, abandoned the clear upper layer, and then after repeated piping and drumming for single cell suspension with fresh special MSCs medium from the mice bone marrow, inoculated in culture dish; The medium was changed after 72 hours and removal of nonadherent cells. Changed once every two days after that. When the colony in the culture dish were fused, and when the density of cells was about 70%, were cultured to mark as P1 generation. After MSCs were passed to P5 generation, to prepare stem cell agent for anti-hypoxia injury with the stem cells were put into Fsl1 over-expression lentivirus with the culture medium; Preferably, inoculated in culture dish according to the density of 3×10⁵/cm².

Preferably, the stem cells were put into Fsl1 over-expression lentivirus with the culture medium change after 12 hours, obtained the stem cell agent for anti-hypoxia injury; More preferably, the culture time can be 72 hours or longer after changed, the culture for a period of time to obtain more stem cell for anti-hypoxia injury.

The invention also discloses a stem cell agent for anti-hypoxia injury with the preparation method of the stem cell agent for anti-hypoxia injury in the above; the application of Fsl1 over-expression lentivirus in improving the survival rate of stem cell transplantation; or the application of Fsl1 over-expression lentivirus in the preparation of stem cell agent for anti-hypoxia injury.

### Beneficial Effects

It can judge the cell infection efficiency by observing the mCherry fluorescence under the inverted fluorescence microscope, and it can be found that most of the cells display strong mCherry fluorescence, indicating that the lentivirus infection is successful.

The stem cell agent for anti-hypoxia injury prepared by the invention, the efficiency of the cells infecting was confirmed with mCherry fluorescence by inverted fluorescence microscope, and it can be found that the most of the cells were shown strong mCherry fluorescence light, suggested that the lentivirus infection was successful.

As a kind of primary adult stem cells with abundant sources and easy access, MSCs are widely used in regenerative medicine as seed cells for cell transplantation due to its excellent tissue repair ability. However, the existing MSCs have the problem of weak anti hypoxia damage, which leads to the fact that the repair effect of stem cells is far lower than the theoretical design. The invention can obviously improve the anti hypoxia damage ability of stem cells after transforming stem cells by using FSTL1, so as to improve the transplantation survival rate. Therefore, the invention also discloses the application of FSTL1 in improving the survival rate of stem cell transplantation.

The prior art has studied the effect of Fsl1 on myocardial infarction, but there is no literature related to the use of Fsl1 to transform stem cells, and there is no report on the effect of Fsl1 transforming stem cells; the invention provides that the MSCs modified by FSTL1 have excellent anti hypoxia damage ability and can improve the survival rate of transplantation. Therefore, the invention also discloses the application of Fsl1 in the preparation of stem cell agent for anti-hypoxia injury The invention also discloses the application of preparing the stem cell agent for anti-hypoxia injury in the preparation of stem cell agent for anti-hypoxia injury system or improving the survival rate of stem cell transplantation. At the same time, the invention discloses the application of stem cell agent for anti-hypoxia injury in the preparation of drugs or health products for preventing or treating myocardial infarction. Preparing the stem cell agent for anti-hypoxia injury of the invention has the ability to resist the local poor hypoxia microenvironment of myocardial infarction, and can become an important means of regulating cardiac homeostasis and pathological remodeling.

### DRAWINGS

FIG. 1 is a morphological identification of mice MSCs from the mice bone marrow;

FIG. 2 is a flow characterization of mice MSCs from the mice bone marrow;

FIG. 3 shows a significant decrease in Fstl1 expression in MSCs caused by persistent hypoxia stimulation;

FIG. 4 shows that both MSCs-mCherry and MSCs-Fstl1 strongly express mCherry fluorescence;

FIG. 5 is a flow characterization of MSCs-mCherry and MSCs-Fstl1;

FIG. 6 is a high expression of MSCs-Fstl1 and high secretion of Fstl1;

FIG. 7 shows that MSCs-Fstl1 is more effective against apoptosis induced by hypoxia;

FIG. 8 shows that MSCs-Fstl1 has stronger cell proliferation ability in anoxic environment;

FIG. 9 shows that MSCs-Fstl1 has better cell viability under normoxia and anoxic conditions;

FIG. 10 shows that MSCs-Fstl1 cell transplantation significantly improves cardiac function after myocardial infarction (M-mode ultrasound);

FIG. 11 shows that MSCs-Fstl1 cell transplantation significantly promotes EF, FS, LVID; d and LVPW

FIG. 12 is a MSCs-Fstl1 cell transplantation to reduce the area of myocardial infarction (Mason staining schematic);

FIG. 13 shows the effective reduction of myocardial infarction area (statistical results) by MSCs-Fstl1 cell transplantation;

FIG. 14 is a graph showing the retention of transplanted cells in hypoxic myocardium by mCherry autofluorescence and immunofluorescence co-localization;

FIG. 15 is a graph of transplanted cells labeled with DiI to assess the resident of transplanted cells in hypoxic myocardium.

### EMBODIMENTS OF THE INVENTION

Following examples are provided to assist those skilled in the art in a more complete understanding of the invention, but are not intended to limit the invention in any way. In the present invention, the medium was changed is that the virus infection liquid was complete replaced with normal cell culture medium complete cell culture medium.

### Example 1 Preparation and Performance Test of Stem Cell Agent for Anti-Hypoxia Injury

The main materials and sources used are as follows:

C57BL/6J mice (Joinn Laboratories, Inc, approved by the Ethics Committee of Soochow University); DMEM/F12 medium (Gibco, USA); special MSCs medium from the mice bone marrow (cyagen, China); Trypsin (Sigma, USA); Ultra Clean Bench (Antai, China); Carbon Dioxide Incubator (Thermo, USA); table centrifuge (Thermo, USA); Flow Cytometry (Millipore, USA); Inverted Fluorescence Microscope (ZEISS, Germen); Nanodrop2000 ultra-micro spectrophotometer (Thermo, USA); real-time PCR instrument (ABI, USA); full-band multi-function microplate reader (BIO-TEK, USA); reverse transcription kit (Takara, Japan); FITC-CD29 antibody (Biolegend, USA); APC-CD44 antibody (Biolegend, USA); FITC-CD90 antibody (Biolegend, USA); PE-CD45 antibody (Biolegend, USA); APC-CD117 antibody (Biolegend, US); Fstl1 ELISA Test Kit (R&D, USA); Click-iT Plus EdU Alexa Fluor 647 Flow Test Kit (Life, USA); Annexin V-FITC Reagent (BD, USA); CCK-8 kit (Dojindo, Japan); Fstl1 over-expression lentivirus (LV-Fstl1) and control (LV-mCherry) (Genechem, China)

1.1 Obtaining MSCs from the Mice Bone Marrow

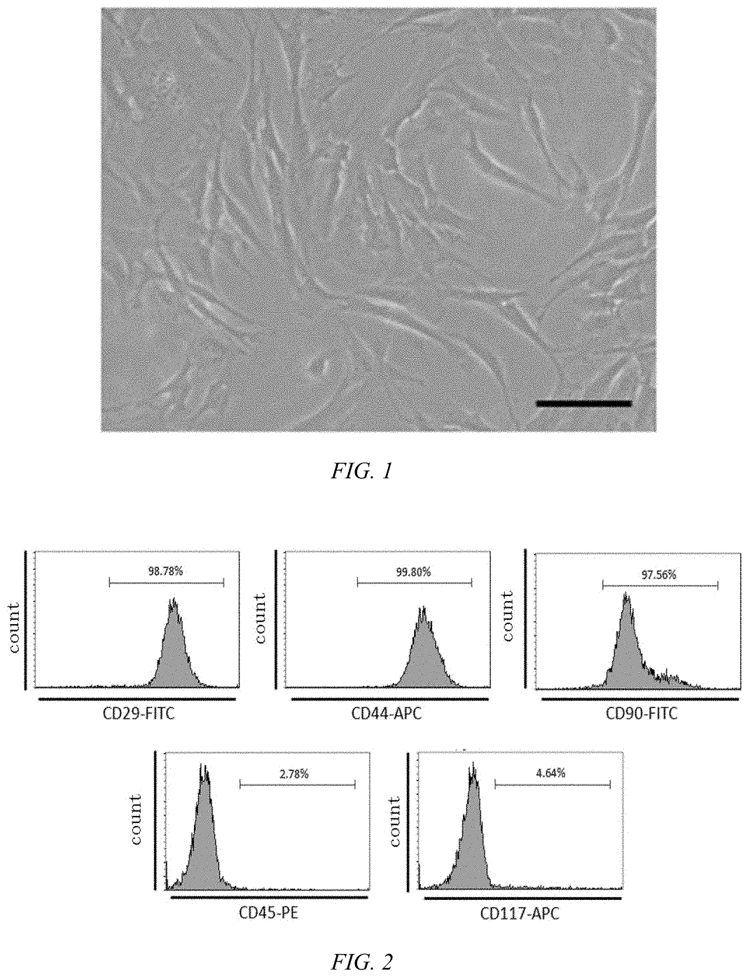

Selected the male mice C57BL/6J at 2-3 weeks old, which killed by cervical dislocation. Then mice were immersed in 75% alcohol for 10 minutes. Under sterile conditions, removed the tow lower limbs, which were immersed in serum free basic medium DMEM/F12. Remove the surface muscles of femur and tibia by scissors and tweezers; Imbibed with 1 ml injector the serum free basic medium DMEM/F12 which were added penicillin and streptomycin, and then washed the marrow cavity; After washed the marrow all out, centrifugation, abandoned the clear upper layer, and then after repeated piping and drumming for single cell suspension with fresh special MSCs medium from the mice bone marrow, according to the density of 3×10⁵/cm² inoculated in culture dish; The medium was changed after 72 hours and removal of nonadherent cells. Changed once every two days after that. When the colony in the culture dish were fused, and when the density of cells was about 70%, were cultured to remark P1. FIG. 1 shown the P6 generation MSCs. It can be seen that the MSCs from the mice bone marrow under the light microscope were evenly distributed and uniform in morphology. They were fibroblast-like or flat, and a few was fusiform, with protrusions of varying lengths and uneven thickness.

1.2 Identification of Mice MSCs from the Mice Bone Marrow by Flow Cytometry

When the cell confluence reached 80%, conventional trypsin was digested, regulated cell concentration to 5×10⁶ cells/ml; Putted FITC-CD29 antibody, APC-CD44 antibody, FITC-CD90 antibody, PE-CD45 antibody and APC-CD117 antibody, respectively. Incubated at 4° C. for 30 min; washed with PBS and measure cell surface markers by flow cytometry. FIG. 2 is a graph showing the results of flow cytometry. Mice MSCs from the mice bone marrow express CD29, CD44 and CD90, and do not express CD45 and CD117.

1.3 Sustained Hypoxia Stimulation Caused a Significant Decrease in Fstl1 Expression in MSCs

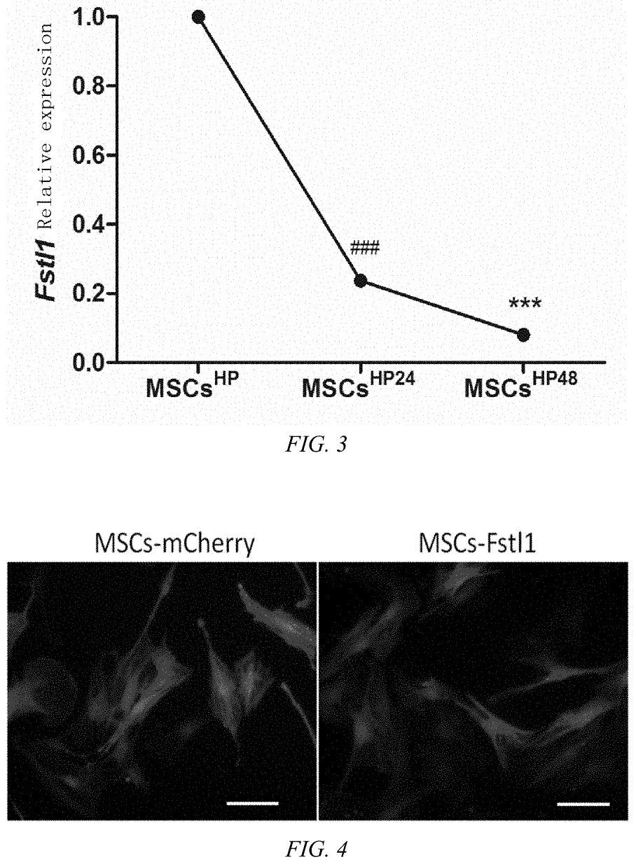

When the confluence of MSCs reached 70%, continuous hypoxia stimulation (1% 02) was performed, and cells were harvested at 0 h, 24 h and 48 h, and total RNA, inversion and qRT-PCR were extracted. Four replicate wells were set for each sample, and the reaction system was 10 μl with GAPDH as an internal reference. The primer sequences used were as follows: Fstl1: 5-TTATGATGGGCACTGCAA-3 (SEQ ID NO: 1) and 5-ACTGCCTTTAGAGAACCAG-3 (SEQ ID NO: 2); GAPDH: 5-TGCCCAGAACATCATCCCT-3 (SEQ ID NO: 3) and 5-GGTCCTCAGTGTAGCCCAAG-3 (SEQ ID NO: 3). FIG. 3 shows that continuous hypoxia stimulation caused a significant down-regulation of Fstl1 expression in MSCs, suggesting that Fstl1 may play an important role in the protection of hypoxic injury. ###P<0.001 (0 h vs 24 h); ***P<0.001 (0 h vs 48 h).

1.4 Obtaining Anti-Hypoxic Damage Stem Cell Preparation

In step 1, when the confluence of MSCs reaches 50%, the number of virus particles is converted according to the multiplicity of infection (MOI)=10; respectively, the Fstl1 Over-expression lentivirus (LV-Fstl1) solution (Jikai) and corresponding Empty control virus solution (LV-mCherry), and add polybrene (8 μg/ml) to increase the efficiency of virus infection; 37° C., 5% CO₂ incubator infection overnight; 12 h after the virus solution and normal medium added; 72 h later The fluorescence intensity of mCherry was observed under inverted fluorescence microscope to determine the cell infection efficiency. The high expression and secretion of Fstl1 were detected by qRT-PCR and ELISA. The MSCs were identified by flow cytometry. Above, a stem cell preparation resistant to hypoxia is obtained, which is called MSCs-Fstl1, and the corresponding control is called MSCs-mCherry.

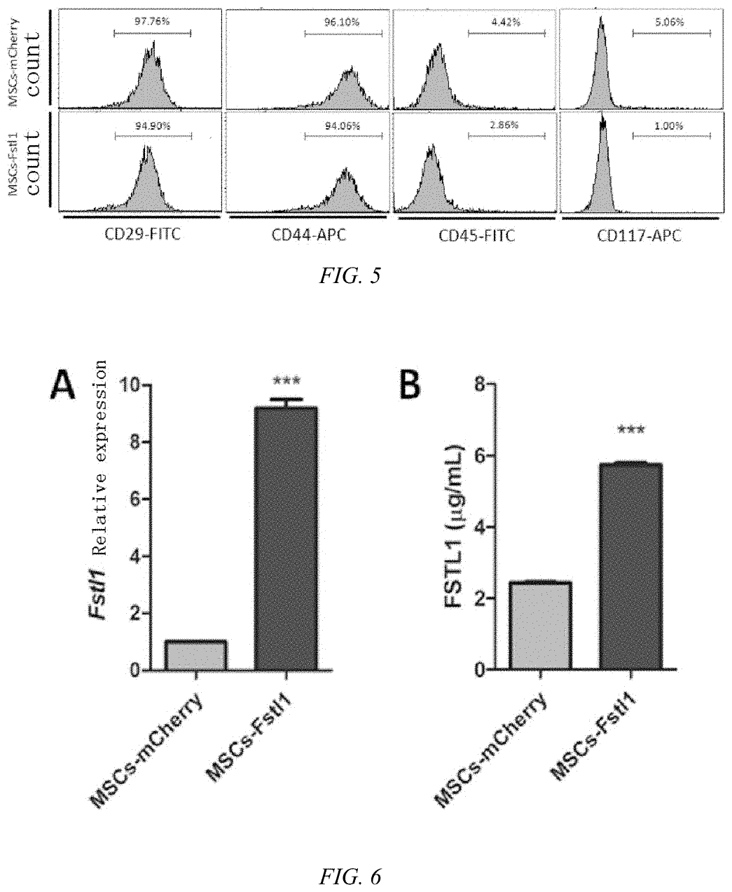

FIG. 4 is a fluorescence diagram of MSCs-mCherry and MSCs-Fstl1. The cells in MSCs-mCherry and MSCs-Fstl1 showed strong mCherry fluorescence, and still retained the typical morphology of MSCs, which proved that lentivirus infection was successful. FIG. 5 shows MSCs-mCherry And MSCs-Fstl1 flow cytometry results, MSCs-mCherry and MSCs-Fstl1 both expressed CD29 and CD44, and did not express CD45 and CD117.

1.5 Identification of Fstl1 Transcription Level in MSCs-Fstl1 Cells by qRT-PCR

MSCs-mCherry and MSCs-Fstl1 total RNA were routinely extracted, and the Fstl1 transcription level was determined as in Example 1.3. As shown in FIG. 6A, the Fstl1 transcription level of MSCs-Fstl cells was increased to 9.18 times that of the control MSCs-mCherry group, ***P<0.001.

1.6 Identification of Fstl1 Secretion Level in MSCs-Fstl1 Cells by ELISA

The Fstl1 capture antibody was coated with a 96-well microtiter plate to prepare a solid phase carrier, and the specimen (MSCs-mCherry or MSCs-Fstl1 supernatant) or the standard, the biotinylated Fstl1 detection antibody, and the HRP-labeled avidin were sequentially added. After thorough washing, the substrate was developed with color. The color depth is positively correlated with the Fstl1 content of the sample. The absorbance (OD value) was measured with a microplate reader at a wavelength of 450 nm, and the concentration of Fstl1 in the sample was calculated from the standard curve.

FIG. 6B shows that the Fstl1 concentration of the supernatant of MSCs-Fstl1 cells was 12.44 times that of the control group, demonstrating that MSCs-Fstl1 can achieve high secretion of Fstl1, ***P<0.001.

1.7 Annexin V Labeling for Detection of MSCs-Fstl1 Resistance to Hypoxia-Induced Apoptosis

MSCs-mCherry and MSCs-Fstl1 were inoculated in a 12-well plate at 8×10⁴/well, and the cells were replaced with fresh medium after overnight adherence, and the hypoxia stimulation (1% O₂) was continued for 48 H, with 0.25% without EDTA. The cells were trypsinized and collected, washed twice with PBS, and resuspended in 100 μl of Annexin V labeling buffer at a cell concentration of approximately 10⁶ cells/ml. Add 5 μl of Annexin V-FITC and mix at room temperature for 15 min in the dark, and test by flow cytometry.

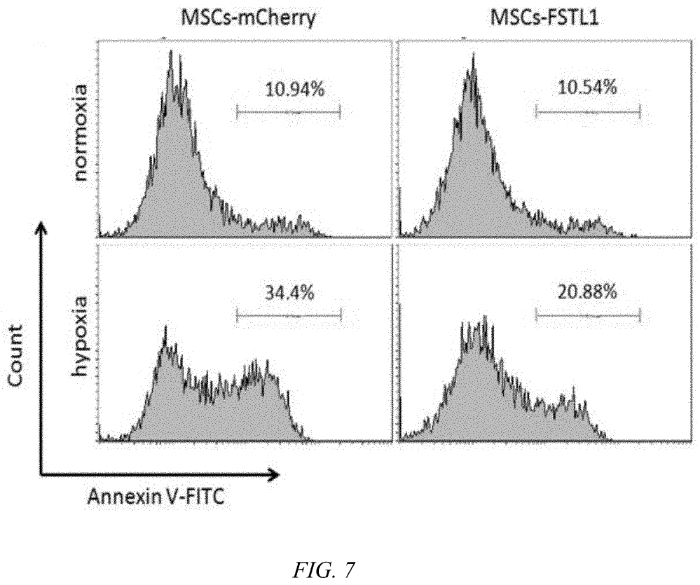

The Annexin V staining experiment of FIG. 7 showed that the proportion of Annexin V+ cells in the MSCs-Fstl1 group was significantly lower than that in the MSCs-mCherry group under hypoxia stimulation.

1.8 EdU Labeling Detects Proliferation of MSCs-Fstl1 in Hypoxic Environment

MSCs-mCherry and MSCs-Fstl1 were inoculated in a 12-well plate at a certain density. After adherence, the cells were subjected to continuous hypoxia stimulation (1% 02) for 48 H, and added with EdU (10 μM) for 1 h at 37° C. Trypsin digestion and collection. The cells were washed twice with PBS, fixed, fluorescently infiltrated, and detected by flow cytometry.

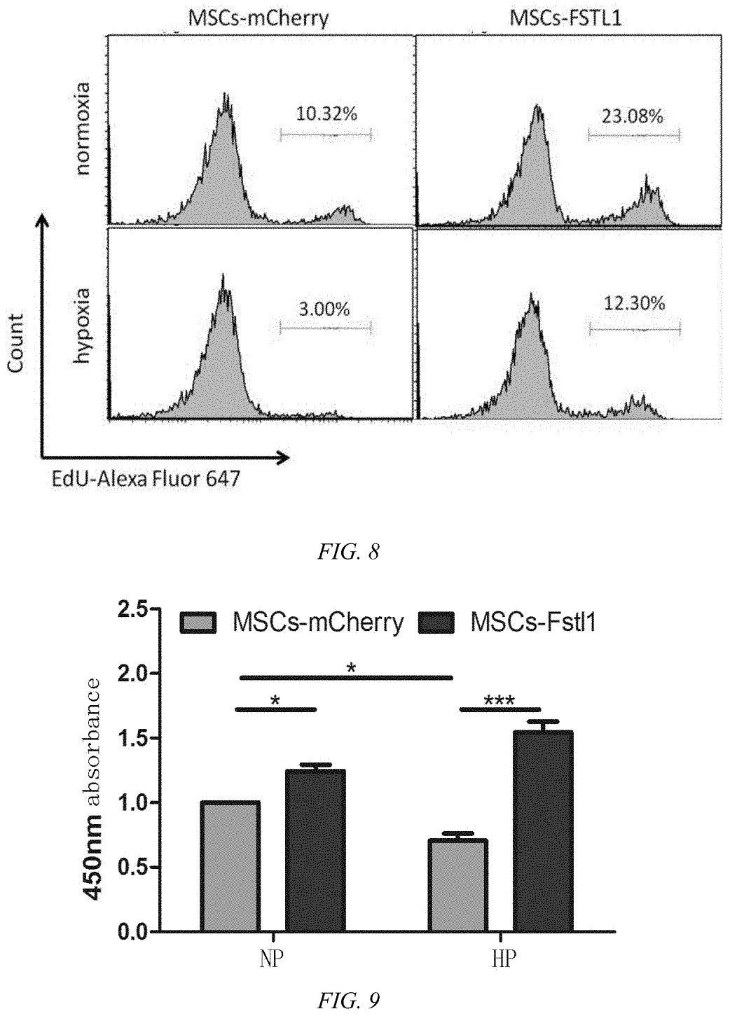

The results of EdU infiltration experiments in FIG. 8 showed that the proliferation ability of MSCs-Fstl1 group was higher than that of MSCs-mCherry group under normoxia and hypoxia.

1.9 CCK-8 Assay for Cell Viability of MSCs-Fstl1 in Hypoxic Environment

MSCs-mCherry and MSCs-Fstl1 were plated at a density in 96-well plates. After adherence, continuous hypoxia stimulation (1% O₂) for 48 H, 100 μl of fresh medium and 10 μl of CCK-8 reaction solution per well, and absorbance at 450 nm per 0.5 h between 0.5 and 2 h using a microplate reader. .

The CCK-8 experiment results in FIG. 9 show that the cell viability of MSCs-Fstl1 under normal and hypoxic conditions is 1.24 and 2.19 times that of MSCs-mCherry, respectively, *P<0.05, **P<0.01, ***P<0.001.

### Example 2 Stem Cell Preparation Against Hypoxia Injury Effectively Improves Cardiac Function after Myocardial Infarction and has Better Resident Effect

The main materials and sources used are as follows:

C57BL/6J mice (Joinn Laboratories, Inc, approved by the Ethics Committee of Suzhou University); Small Animal Ventilator (Alcott Bio, Shanghai); Surgical Instruments (Six Six Vision, Suzhou); Sewing Needle (Gold) Huan Medical, Shanghai); Small Animal Ultrasound Imaging System (Visual Sonics Vevo 2100); Inverted Fluorescence Microscopy (ZEISS, Germany); Masson Staining Kit (Sigma, USA); mCherry Antibody (Abeam, USA); FITC-labeled goat anti-Rabbit IgG secondary antibody (Senta Cruz, USA); CM-DiI (Invitrogen, USA)

2.1 Establishment of a Mice Myocardial Infarction Model

Approximately 25 g of C57BL/6J male mice were used as experimental subjects, and a left anterior descending artery (LAD) ligation method was used to make a myocardial infarction model. After anesthesia was injected intraperitoneally, the patient was intubated by an oral tube and connected to an air ventilator. The respiratory rate was 110 beats/min, the tidal volume was 3 ml, and the respiratory ratio was 1:1.3. In the right lateral position, the left chest longitudinal incision cuts the outer skin, peels off the pectoralis major muscle, and the third and fourth intercostal transverse incision opens the chest, exposes the heart, and tears the happy capsule with tweezers. The left coronary artery is seen to travel roughly by means of a surgical microscope. At the lower edge of the left atrial appendage, about 1 to 2 mm, the LAD was ligated together with a small amount of myocardial tissue. The depth of the needle was about 1 mm and the width was controlled within 3 mm. Close the chest layer by layer. The sham operation group (sham) only passed through the LAD without tying, and the rest were the same model group; after ligation, the ligature to the apex became white, and after 7 days, the left ventricular tissue was stained for cardiac tissue, and obvious fibrosis was observed. Prove that the myocardial infarction model was established successfully.

2.2 Myocardial Injection of Stem Cell Preparation Against Hypoxia Injury

The anti-hypoxic-damaged stem cell preparation MSCs-Fstl1 is mixed with the dispersion medium PBS to obtain a drug for preventing or treating myocardial infarction. After LLA ligation according to the method of step 1 above, the drug was injected into the lower left and lower right sites near the ligation site. The amount of MSCs per mice was 5×10⁵/20 μl, 10 μl per site, with PBS as the PBS. Negative control. Choose the right angle to avoid injection into the left ventricular cavity. A slight lightening of the myocardium indicates that the solution has entered the infarcted ventricular wall.

2.3 Ultrasound Detection of Cardiac Function after Myocardial Infarction

7 days after myocardial infarction, the mice were anesthetized (the method is the same as before), and the left lateral position was removed after the hair was removed. The probe of the cardiac ultrasonic diagnostic apparatus was placed on the anterior wall of the heart, and the left ventricular two-dimensional short axis view was taken at the level of the papillary muscle. M-scan was recorded, and left ventricular ejection fraction (EF), fractional shortening (FS), and left ventricular internal diameter at end-diastole (LVID; d) were measured for 3 consecutive cardiac cycles. And left ventricular posterior wall thickness at end-diastole (LVPW; d).

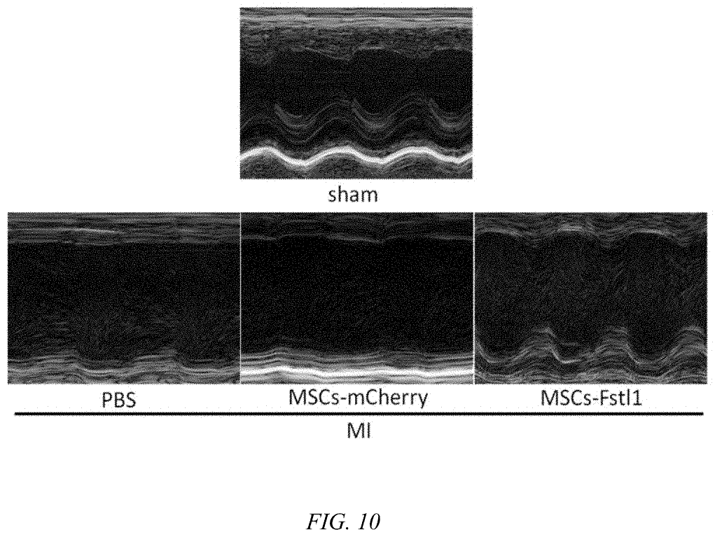

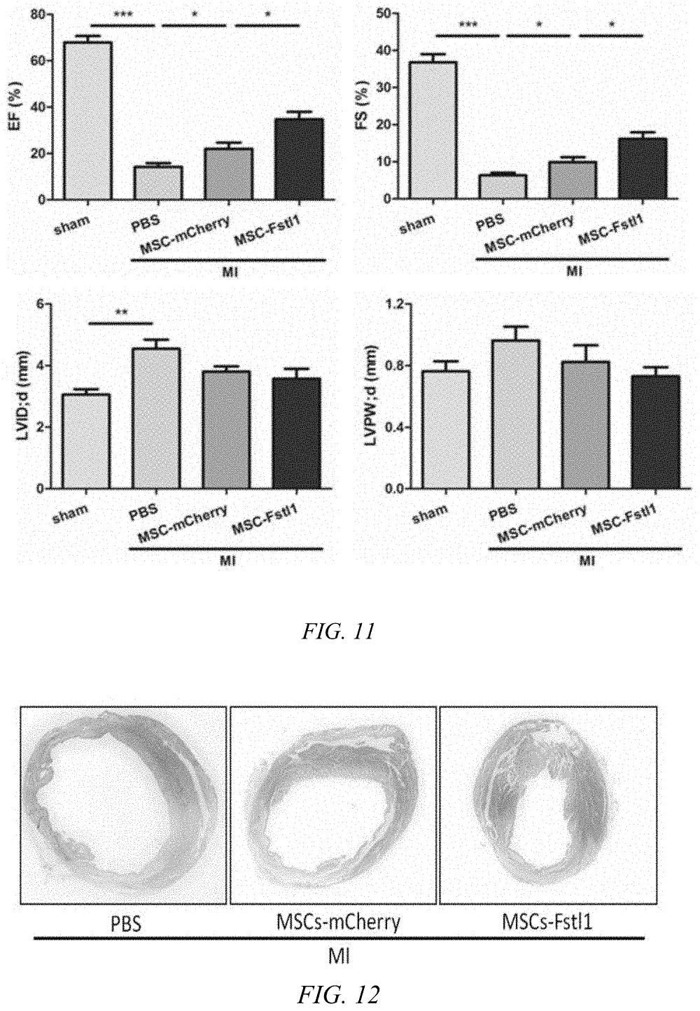

Referring to FIG. 10 and FIG. 11, 7 days after myocardial infarction/transplantation, the cardiac function of mice in the PBS group alone was completely consistent with the characteristics of echocardiography after typical myocardial infarction, EF and FS were significantly decreased, LVID; d was significantly increased, suggesting heart The ventricular remodeling was obvious after the stalk. The cardiac function of the MSCs-mCherry group was significantly better than that of the PBS alone group. The MSCs-Fstl1 group had the best effect on improving cardiac function after myocardial infarction, and the difference was significant compared with the other groups. *P<0.05, **P<0.01, ***P<0.001.

2.4 Masson Staining for Assessment of Myocardial Infarct Size

Mice were sacrificed 7 days after myocardial infarction, and left ventricular tissue was taken for routine tissue section and Masson staining. The area was observed and photographed by a common optical microscope, and the image analysis software Image J was used to analyze the area of each part. The reference formula for calculating the area of myocardial infarction is as follows:

Area of myocardial infarction (%)=actual myocardial infarct area/actual heart cross-sectional area;

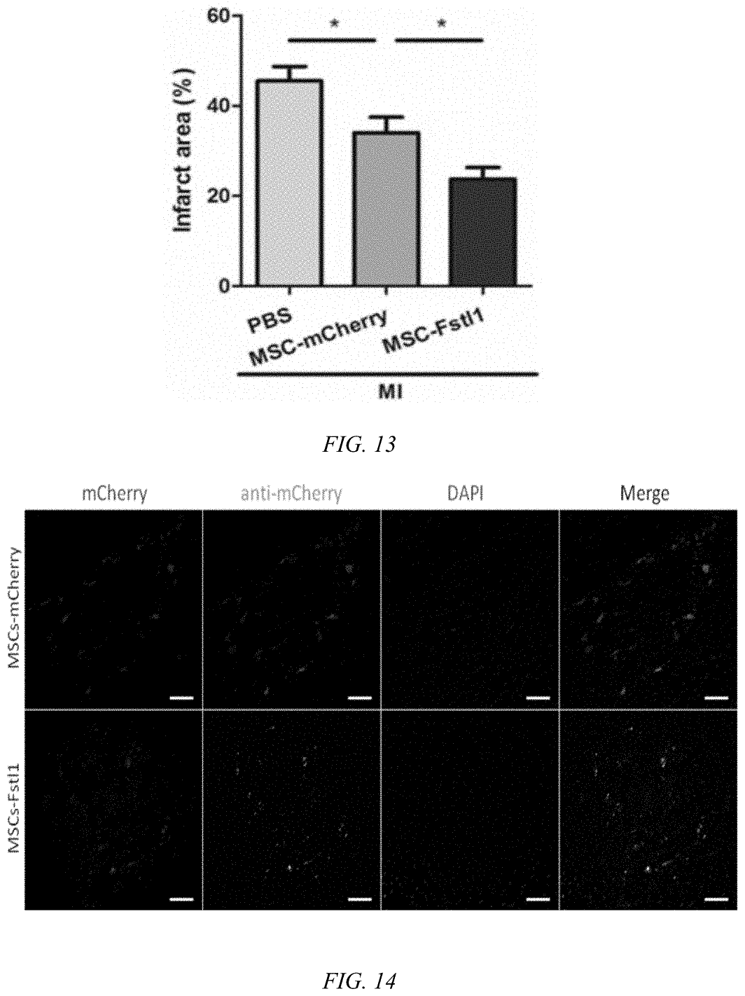

Referring to FIG. 12 and FIG. 13, the infarct size at 7 days after myocardial infarction was observed by Masson staining. The myocardial infarction area of MSCs-mCherry group and MSCs-Fstl1 group was 74.75% and 52.06%, respectively, of PBS group; MSCs—The myocardial infarct size was the lowest in the Fstl1 group, which was 69.64% in the MSCs-mCherry group; *P<0.05. FIG. 12 shows a representative picture in each set of samples, the scale showing 1 mm.

2.5 mCherry Immunofluorescence Assessment of MSCs-Fstl1 Resident

The mice were sacrificed 1 day after cell transplantation and myocardial infarction. The left ventricular tissue was taken for frozen sectioning. The mCherry immunofluorescence staining was performed according to the routine procedure. FITC-labeled goat anti-rabbit IgG secondary antibody was added, and the anti-fluorescence attenuation preparation containing DAPI was used. The agent was sealed and observed simultaneously with a fluorescence microscope and photographed mCherry's own signal (red), FITC signal (green) and DAPI signal (blue).

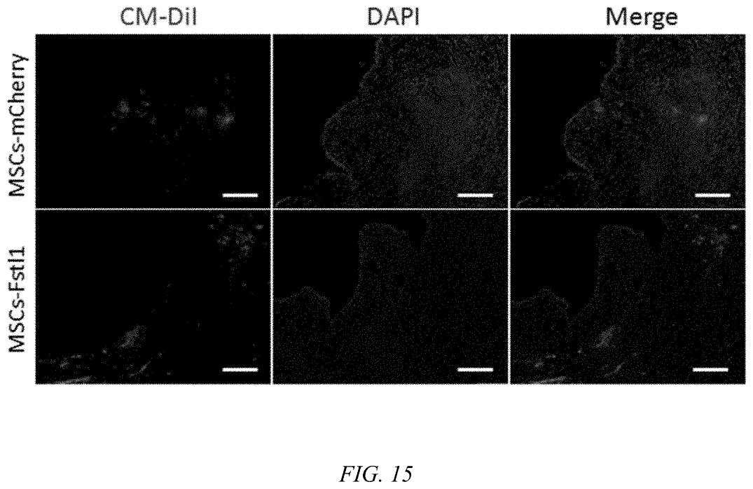

Referring to FIG. 14, the fluorescence of each group was observed under a fluorescence microscope, and it was found that the cell retention of the MSCs-Fstl1 group was significantly higher than that of the MSCs-mCherry control group. The photo shows a representative image from each set of samples, with a scale showing 50 μm.

2.6 CM-DiI Labeled MSCs-Fstl1 to Assess its Resident in Hypoxic Heart

Digest and collect MSCs-mCherry and MSCs-Fstl1, resuspend MSCs in CM-DiI staining solution (1 μg/ml), Incubated for 5 min at 37° C., Incubated for 15 min at 4° C., washed twice with PBS, perform myocardial infarction surgery and Local myocardial injection. The mice were sacrificed 3 days after transplantation, and the tissues near the injection site were taken for routine sectioning. The CM-DiI signal of the local cell transplantation area was observed by fluorescence microscope.

Referring to FIG. 15, the CM-DiI fluorescence of each group was observed under a fluorescence microscope. The CM-DiI signal region of the MSCs-Fstl1 group was significantly higher than that of the MSCs-mCherry control group, suggesting that MSCs-Fstl1 resides in hypoxic myocardium. Better than MSCs-mCherry, the scale shows 200 μm.

The above description is only a preferred embodiment of the present invention, and it should be noted that those skilled in the art can also make several improvements and retouchings without departing from the principles of the present invention. It should be considered as the scope of protection of the present invention.

1. A method of preparing a stem cell agent for anti-hypoxia injury comprising the following steps:
mixing stem cells and an Fsl1 over-expression lentivirus in a culture medium,
replacing the culture medium after 10-15 hours, and
obtaining the stem cell agent for anti-hypoxia injury.

2. A method of preparing a drug for preventing and treating of acute myocardial infarction, comprising the following steps:
mixing stem cells and an Fsl1 over-expression lentivirus in a culture medium;
replacing the culture medium after 10-15 hours;
obtaining a stem cell agent for anti-hypoxia injury;
mixed the stem cell agent for anti-hypoxia injury with a dispersion medium to obtain the drug for preventing or treating acute myocardial infarction.

3. The method according to claim 1, wherein the stem cells and the Fsl1 over-expression lentivirus are mixed in the presence of a polybrene in the culture medium.

4. The method according to claim 1, wherein the stem cells are mice bone marrow derived mesenchymal stem cells (MSC), and an amount of Fsl1 over-expression lentivirus is calculated based on MOI=10.

5. The method according to according to claim 1, further comprising:
selecting male mice C57BL/6J at 2-3 weeks old, sacrificed by cervical dislocation;
immersing the mice in 75% alcohol for 10 minutes;
removing, under sterile conditions, two lower limbs and immersing in serum free basic medium DMEM/F12;
removing surface muscles of femur and tibia;
imbibing a serum free basic medium DMEM/F12 with penicillin or streptomycin with a 1 ml injector and washing a marrow cavity to collect a bone marrow;
centrifuging the bone marrow and discarding a clear upper layer;
adding the bone marrow to a fresh special MSCs medium to form a single cell suspension;
inoculating the single cell suspension in a culture dish;
replacing a culture medium after 72 hours and removing nonadherent cells;
replacing the culture medium once every two days thereafter;
marking a colony in the culture dish P1 generation when the colony is fused and a density of cells is about 70%;
mixing with the Fsl1 over-expression lentivirus to obtain the stem cell agent for anti-hypoxia injury when the colony reaches P5 generation.

6. The method according to claim 1, wherein the culture medium is DMEM/F12, and the culture medium is replaced after 12 hours.

7. A stem cell agent for anti-hypoxia injury prepared according the method of claim 1.

8. The method according to claim 2, wherein the stem cells and the Fsl1 over-expression lentivirus are mixed in the presence of a polybrene in the culture medium.

9. The method according to claim 2, wherein the stem cells are mice bone marrow derived mesenchymal stem cells (MSC), and an amount of Fsl1 over-expression lentivirus is calculated based on MOI=10.

10. The method according to according to claim 2, further comprising:
selecting male mice C57BL/6J at 2-3 weeks old, sacrificed by cervical dislocation;
immersing the mice in 75% alcohol for 10 minutes;
removing, under sterile conditions, two lower limbs and immersing in serum free basic medium DMEM/F12;
removing surface muscles of femur and tibia;
imbibing a serum free basic medium DMEM/F12 with penicillin or streptomycin with a 1 ml injector and washing a marrow cavity to collect a bone marrow;
centrifuging the bone marrow and discarding a clear upper layer;
adding the bone marrow to a fresh special MSCs medium to form a single cell suspension;
inoculating the single cell suspension in a culture dish;
replacing a culture medium after 72 hours and removing nonadherent cells;
replacing the culture medium once every two days thereafter;
marking a colony in the culture dish P1 generation when the colony is fused and a density of cells is about 70%;
mixing with the Fsl1 over-expression lentivirus to obtain the stem cell agent for anti-hypoxia injury when the colony reaches P5 generation.

11. The method according to claim 2, wherein the culture medium is DMEM/F12, and the culture medium is replaced after 12 hours.

12. A drug for preventing and treating acute myocardial infarction prepared according the method of claim 2.


SEQUENCE LISTING (4 sequences)

SEQ ID NO: 1 - DNA, 18 bp, Artificial Sequence
  Primer
ttatgatgggcactgcaa

SEQ ID NO: 2 - DNA, 19 bp, Artificial Sequence
  Primer
actgcctttagagaaccag

SEQ ID NO: 3 - DNA, 19 bp, Artificial Sequence
  Primer
tgcccagaacatcatccct

SEQ ID NO: 4 - DNA, 20 bp, Artificial Sequence
  Primer
ggtcctcagtgtagcccaag